(19) United States
(12) Patent Application Publication
(10) Pub. No.: US 2011/0245811 A1
(43) Pub. Date: Oct. 6, 2011

(54) Handy scratcher

(76) Inventor: Joycelyn Sabal, Brooklyn, NY (US)

(21) Appl. No.: 13/065,593

(22) Filed: Mar. 24, 2011

Related U.S. Application Data
(60) Provisional application No. 61/341,006, filed on Mar. 25, 2010.

(51) Int. Cl. A61B 17/00

(52) U.S. Cl. 606/1

(57) ABSTRACT

The Handy Scratcher is essentially tubular in shape. The proximal end of the Handy Scratcher, where it is held, consists of a rubberized grip to facilitate a secure hold while the unit is in use. In addition, a sliding clip mechanism is featured, which serves to extend and retract the scratching component. As such, the latter features a three-tier connection system, which allows it to be extended outward from and retracted into the housing. The distal end of the Handy Scratcher is the scratcher itself. A thin, hard plastic component, the scratcher is approximately the size of a cotton swab, small enough to sufficiently attend to an itch without moving a hair out of place. Simple in design yet effective in application, the Handy Scratcher is put to work in a matter of seconds. Before heading out, the user simply places the compact device in a purse or even a pocket. Should she feel an itch, she need only retrieve the Handy Scratcher, extend the distal end, and insert the tip into the hair to scratch. Returning the unit to its storage space, the user feels confident that she has taken care of an annoying itch, and her hair still looks as good as it did when she left the house.

### CLAIM OF PRIORITY

This patent application claims priority under 35 USC 119 (e) (1) from U.S. Provisional Patent Application Ser. No. 61/341,006 filed Mar. 25, 2010, of common inventorship herewith entitled, “The Handy Scratcher.”

### FIELD OF THE INVENTION

The present invention pertains to the field of personal scratching devices, and more specifically to the field of scalp scratching devices.

### BACKGROUND OF THE INVENTION

The prior art has put forth several designs for scalp scratching devices. Among these are:

U.S. Pat. No. 3,768,463 to Darney George Curry describes a device provided for scratching and massaging the scalp, and for combing and lifting the depressed areas of bouffant and afro hair styles. The device includes a manual handle and an elongated thin rigid prong adjustably mounted at one end in the handle and extending axially therefrom. A rounded scalp-engaging tip is provided on the terminal end of the prong. A removable cap is provided which is arranged to be positioned over the elongated prong and engaged with the handle to enclose the prong during non-use.

U.S. Pat. No. 6,382,216 to Wayne Clark describes a hair brush with a body having bristles extending therefrom for brushing hair and a tubular handle connected to the body which handle has a bore disposed normal to its longitudinal axis adjacent to its distal end along with a blind aperture extending for its distal end into the handle, generally along its longitudinal axis; plus a hair pick with a circular opening therein which pick is configured to snugly fit into the blind aperture.

U.S. Pat. No. 6,109,273 to Nancy G. Schleicher describes a dual blade teasing comb and hair pick which includes a laterally space pair of comb blades, each including an elongated spine having a longitudinal series of teeth depending therefrom and an elongated rearwardly extending cylindrical hair.

None of these prior art references describe the present invention.

### SUMMARY OF THE INVENTION

It is an object of the present invention to provide an improved scalp scratching device.

### BRIEF DESCRIPTION OF THE DRAWINGS

FIG. 1 is an elevational angled perspective view of the device of the present invention.

FIG. 2 is a close-up right angled perspective view of a portion of the device of the present invention.

FIG. 3 is a close-up right angled perspective view of a portion of another embodiment of the device of the present invention.

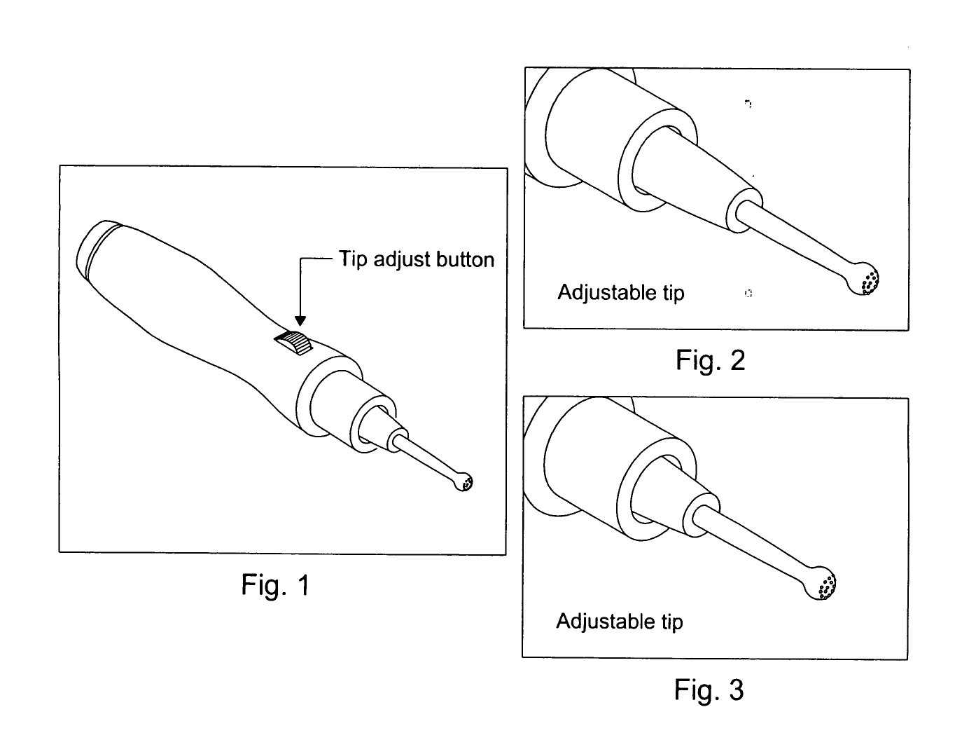

### DETAILED DESCRIPTION OF THE INVENTION

The present invention, hereinafter referred as the Handy Scratcher, is a handheld device that attends to itching scalps, thereby providing female consumers with a simple and effective means of scratching the head without compromising their hairstyle.

The Handy Scratcher is essentially tubular in shape. The proximal end of the Handy Scratcher, where it is held, consists of a rubberized grip to facilitate a secure hold while the unit is in use. In addition, a sliding clip mechanism is featured, which serves to extend and retract the scratching component. As such, the latter features a three-tier connection system, which allows it to be extended outward from and retracted into the housing. The distal end of the Handy Scratcher is the scratcher itself. A thin, hard plastic component, the scratcher is approximately the size of a cotton swab, small enough to sufficiently attend to an itch without moving a hair out of place. Simple in design yet effective in application, the Handy Scratcher is put to work in a matter of seconds. Before heading out, the user simply places the compact device in a purse or even a pocket. Should she feel an itch, she need only retrieve the Handy Scratcher, extend the distal end, and insert the tip into the hair to scratch. Returning the unit to its storage space, the user feels confident that she has taken care of an annoying itch, and her hair still looks as good as it did when she left the house. Easy and safe to use for all ages, the Handy Scratcher is sure to be well received by the vast majority of the female population.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A scalp scratching device, comprising: an essentially tubular handle having a rubberized grip at its proximal end, a sliding clip to extend and retract the telescoping distal end which distal end comprises a thin, hard plastic component, approximately two to three inches in length and terminates in a knob approximately one quarter inch in diameter.

2. The device of claim 1 wherein the knob further comprises an irregular surface.

3. The device of claim 1 wherein the tubular handle is contoured to accommodate a human hand.

4. The device of claim 1 wherein the distal end can be extended and secured in a variety of lengths.
